(19) United States
(12) Patent Application Publication
(10) Pub. No.: US 2014/0014108 A1
(43) Pub. Date: Jan. 16, 2014

(54) OXYGEN DELIVERY DEVICE FOR DIFFUSING GAS FLOW

(71) Applicant: David G. Dillard, Atlanta, GA (US)

(72) Inventor: David G. Dillard, Atlanta, GA (US)

(21) Appl. No.: 13/772,172

(22) Filed: Feb. 20, 2013

Related U.S. Application Data
(60) Provisional application No. 61/672,274, filed on Jul. 16, 2012.

(51) Int. Cl. A61M 16/06; A61M 16/00

(52) U.S. Cl. CPC A61M 16/0666; A61M 16/0057
USPC 128/204.18; 128/207.18

(57) ABSTRACT

A nasal cannula diffuses a gas stream to distribute the volume of the gas stream as it is delivered into a user's nostril. The nasal cannula can direct flow of gas, such as oxygen, away from the more sensitive nasal tissues. The nasal cannula can diffuse the gas flow out of a plurality of orifices formed in the nostril prongs of the nasal cannula device, rather than sending the entire volume of gas out of a single orifice, as is the case with conventional nasal cannula devices.

### CROSS-REFERENCE TO RELATED APPLICATION

This application claims the benefit of priority of U.S. provisional application No. 61/672,274, filed Jul. 16, 2012, the contents of which are herein incorporated by reference.

### BACKGROUND OF THE INVENTION

The present invention relates to oxygen delivery devices and, more particularly, to an oxygen delivery device for diffusing gas flow and thus reducing nasal irritation and bleeding.

Nasal oxygen delivery often results in nasal drying and frequent bleeding due to the orientation of an oxygen stream directed toward more delicate nasal tissues and the oxygen stream's velocity.

Referring to FIGS. 1A and 1B, current nasal cannula oxygen delivery devices 100 have an outlet 140 at the end of each nasal insertion section (prong) 120 which causes the oxygen flow (which lacks any significant moisture content) to be directed to a single point inside each nostril. Continued oxygen use can cause this gas stream to dry out the nostrils, which is the most common area of bleeding from the nose 110.

As can be seen, there is a need for an oxygen delivery device that can diffuse oxygen gas flow into a user's nostrils to help prevent nostril dryness and bleeding.

### SUMMARY OF THE INVENTION

In one aspect of the present invention, an oxygen delivery device comprises a tubing; a manifold fluidly connected to the tubing; first and second prongs extending from and fluidly connected to the manifold; and a plurality of orifices formed along a length of the first and second prongs.

In another aspect of the present invention, an oxygen delivery device comprises a tubing; first and second prongs extending from and fluidly connected to the tubing; and a plurality of orifices formed along a length of the first and second prongs, wherein terminal ends of the first and second prongs are closed; the plurality of orifices includes from about 4 to about 14 orifices in each of the first and second prongs; each of the plurality of orifices are from about 1.5 to about 2.5 mm in diameter; and the plurality of orifices are spaced apart by a distance from about 1.5 to about 2.5 mm.

In a further aspect of the present invention, a method for preventing injury to nostrils during oxygen delivery by a nasal cannula comprises delivering oxygen through a plurality of orifices formed along a length of first and second prongs fluidly connected to tubing, the first and second prongs inserted into a user's nostrils, wherein terminal ends of the first and second prongs are closed.

These and other features, aspects and advantages of the present invention will become better understood with reference to the following drawings, description and claims.

### BRIEF DESCRIPTION OF THE DRAWINGS

FIG. 1A is a front view illustrating a conventional nasal cannula device in use in a user's nose;

FIG. 1B is a perspective view of a conventional nasal cannula device;

FIG. 2 is a front view illustrating a diffused gas flow nasal cannula device, in use, according to an exemplary embodiment of the present invention;

FIG. 3 is a perspective view of a diffused gas flow nasal cannula device according to an exemplary embodiment of the present invention;

FIG. 4 is a perspective view of a diffused gas flow nasal cannula device according to an exemplary embodiment of the present invention;

FIG. 5 is a top view of the diffused gas flow nasal cannula of FIG. 4; and

FIG. 6 is a front view of the diffused gas flow nasal cannula of FIG. 4.

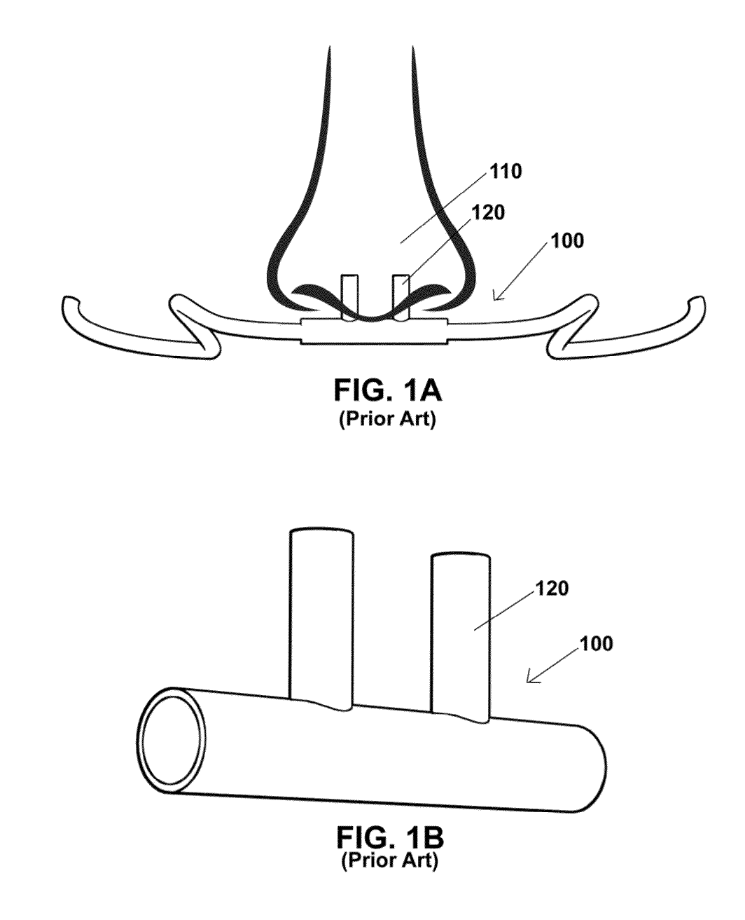

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a nasal cannula that diffuses a gas stream to distribute the volume of the gas stream as it is delivered into a user's nostril. The nasal cannula can direct flow of gas, such as oxygen, away from the more sensitive nasal tissues. The nasal cannula can diffuse the gas flow out of a plurality of orifices formed in the nostril prongs of the nasal cannula device, rather than sending the entire volume of gas out of a single orifice, as is the case with conventional nasal cannula devices.

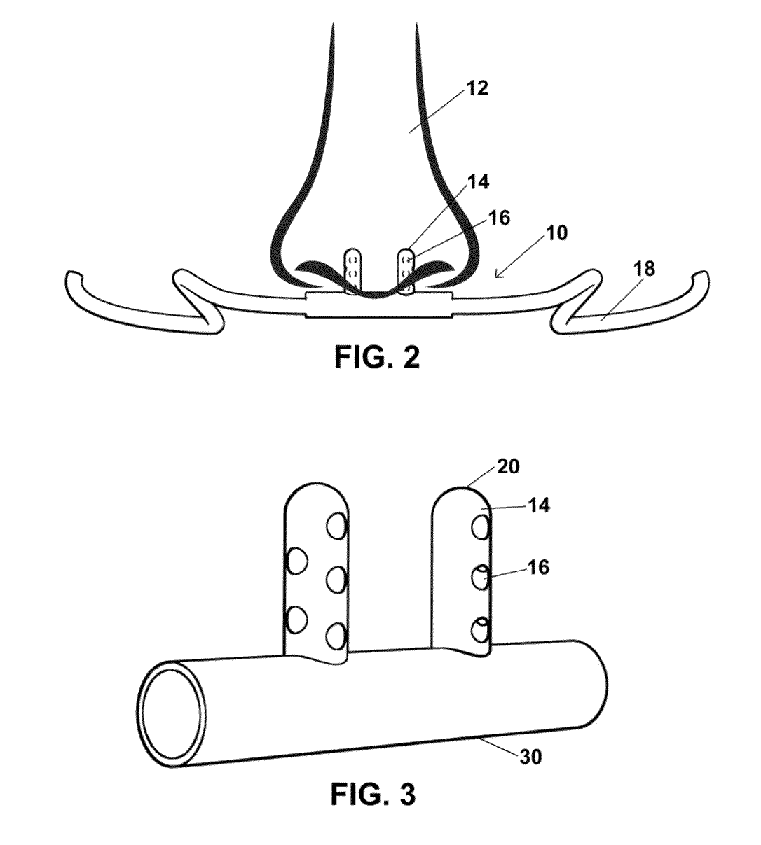

Referring now to FIGS. 2 and 3, a nasal cannula 10 of the present invention uses multiple orifices 16 on the sides of a closed ended tubing (prong) 14 which acts as a manifold for direction of gas out of the multiple orifices 16. The gas exiting the multiple orifices 16 has a distributed volume comparable to the gas exiting the prong ends of a conventional nasal cannula onto a single area within the nose 12. Moreover, the gas exiting the multiple orifices 16 of the nasal cannula 10 of the present invention is directed away from the more sensitive septal mucosa, wherein the gas exiting from the ends of the prongs of conventional nasal cannulas is directed toward this sensitive region within the nose.

A terminal end 20 of each prong 14 of the nasal cannula 10 of the present invention can be closed to gas flow, allowing gas to be vented through the orifices 16 to each anatomically lateral and superior aspect of the nostril. The nasal cannula 10 of the present invention can be designed with tubing 18 similar to conventional nasal cannulas, where the tubing can be looped over a person's ears and secured under the user's chin as is the current medical practice. A base member 30 (also referred to as manifold 30) can be connected to the tubing 18 to permit the gas to be distributed to each of two prongs 14.

The nasal cannula 10 of the present invention can be made from various materials, typically of a polymer material that is molded into a tubular arrangement and attached to the manifold 30 as part of an extrusion process. Orifice ports 16 can be subsequently perforated into the prongs 14 of the cannula device. Alternatively, the nasal cannula 10 may also be molded with the orifice ports 16 pre-formed in the prongs 14.

The above description describes the manifold 30, from which the prongs 14 extend. This manifold 30 can be a separate element to which the tubing 18 is attached or, typically, this manifold 30 can be formed simply from the tubing 18 such that the prongs 14 extend from the tubing 18.

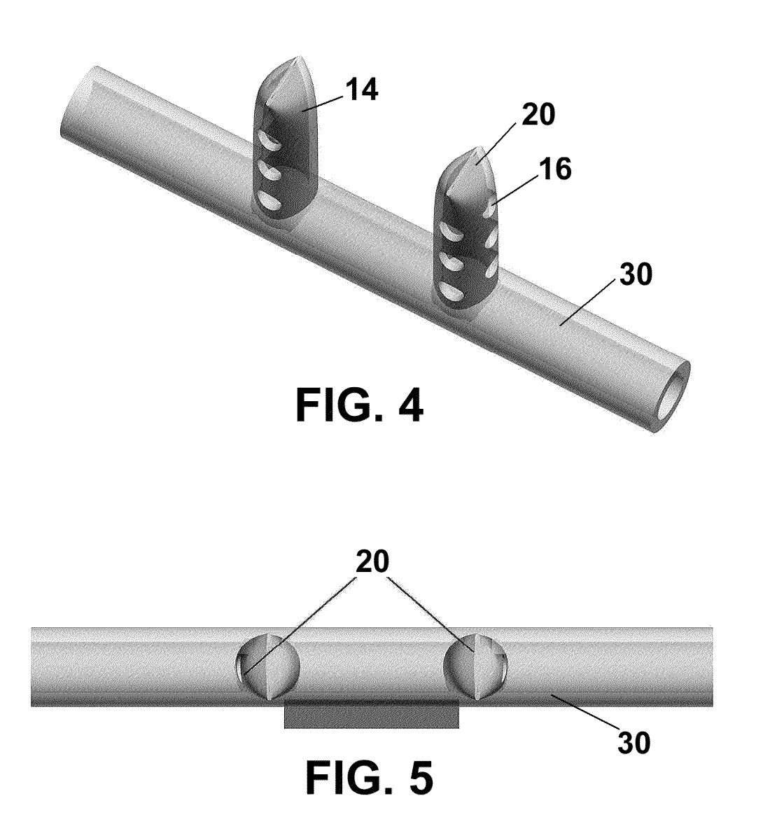

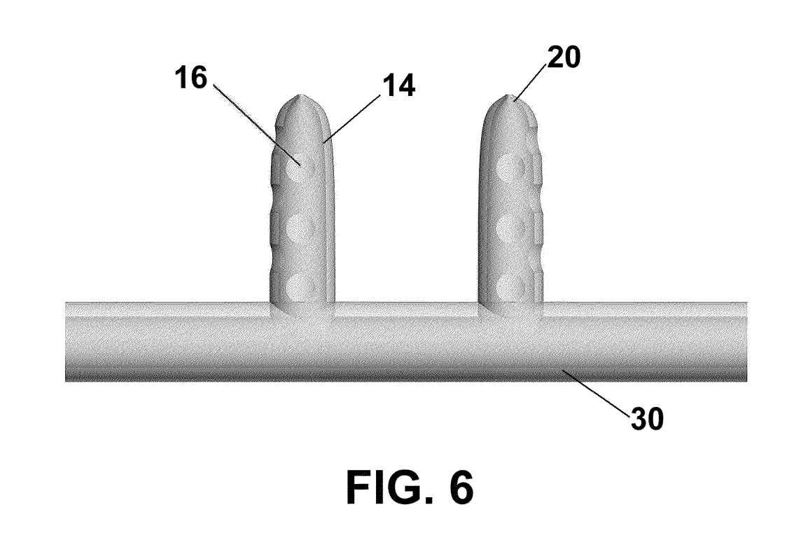

In some embodiments, as shown in FIGS. 4 through 6, the terminal end 20 of each prong 14 can be pinched closed and rounded over. For example, an adhesive can be used to seal the terminal end 20 closed. Other methods, however, can be used to form the closed terminal ends 20 of the prongs 14 of the nasal cannula 10. For example, the terminal ends 20 can be formed closed in the molding/formation process of the nasal cannula prongs.

Each prong of the nasal cannula of the present invention can include from about 3 to about 20 small perforations, typically from about 4 to 14 small perforations formed therethrough. While the Figures show these orifices as round holes, they can be formed in any variety of geometric or non-geometric shapes, including being formed as simple slits or cuts in the prongs to allow gas to escape. In some embodiments, the prongs can be about 12-13 mm in length, with orifices having a diameter from about 1.5 to about 2.5 mm, typically about 2 mm formed along this length, spaced apart by about 1.5 to about 2.5 mm, typically about 2 mm. Three of these orifices can be formed along each of the front and outside faces of each prong, for example, resulting in 6 orifices formed in each prong. The orifices of each face could be offset, as shown in FIGS. 4 and 5, for example. Of course, the above represents an exemplary configuration and size of the orifices along the prongs of the nasal cannula. Variations in the size, shape, quantity and configuration of the orifices are contemplated within the scope of the present invention.

While the drawings show the prongs of the nasal cannula as cylindrical shaped members, the prongs can be formed in various cross-sectional shapes and various exterior configurations, such as having ridges formed about a periphery of the prongs.

It should be understood, of course, that the foregoing relates to exemplary embodiments of the invention and that modifications may be made without departing from the spirit and scope of the invention as set forth in the following claims.

What is claimed is:

1. An oxygen delivery device comprising:
a tubing;
a manifold fluidly connected to the tubing;
first and second prongs extending from and fluidly connected to the manifold; and
a plurality of orifices formed along a length of the first and second prongs.

2. The oxygen delivery device of claim 1, wherein terminal ends of the first and second prongs are closed.

3. The oxygen delivery device of claim 1, wherein the plurality of orifices includes from about 4 to about 14 orifices in each of the first and second prongs.

4. The oxygen delivery device of claim 1, wherein each of the plurality of orifices are from about 1.5 to about 2.5 mm in diameter.

5. The oxygen delivery device of claim 4, wherein the plurality of orifices are spaced apart by a distance from about 1.5 to about 2.5 mm.

6. The oxygen delivery device of claim 1, wherein the plurality of orifices are disposed on a front and outside face of each of the first and second prongs.

7. The oxygen delivery device of claim 1, wherein the manifold is formed in a section of the tubing.

8. An oxygen delivery device comprising:
a tubing;
first and second prongs extending from and fluidly connected to the tubing; and
a plurality of orifices formed along a length of the first and second prongs, wherein
terminal ends of the first and second prongs are closed;
the plurality of orifices includes from about 4 to about 14 orifices in each of the first and second prongs;
each of the plurality of orifices are from about 1.5 to about 2.5 mm in diameter; and
the plurality of orifices are spaced apart by a distance from about 1.5 to about 2.5 mm.

9. The oxygen delivery device of claim 8, wherein the plurality of orifices are disposed on a front and outside face of each of the first and second prongs.

10. A method for preventing injury to nostrils during oxygen delivery by a nasal cannula, the method comprising:
delivering oxygen through a plurality of orifices formed along a length of first and second prongs fluidly connected to tubing, the first and second prongs inserted into a user's nostrils, wherein terminal ends of the first and second prongs are closed.
